(19) United States
(12) Patent Application Publication
(10) Pub. No.: US 2024/0423775 A1
(43) Pub. Date: Dec. 26, 2024

(54) TRANSFER INSTRUMENT AND METHOD FOR OPERATING THE SAME

(71) Applicant: TERUMO KABUSHIKI KAISHA, Tokyo (JP)

(72) Inventor: Koichi HAYAKAWA, Kanagawa (JP)

(73) Assignee: TERUMO KABUSHIKI KAISHA, Tokyo (JP)

(21) Appl. No.: 18/740,901

(22) Filed: Jun. 12, 2024

(30) Foreign Application Priority Data
Jun. 20, 2023 (JP) 2023-100675

(51) Int. Cl. A61F 2/00

(52) U.S. Cl. CPC A61F 2/0063; A61F 2002/0072

(57) ABSTRACT

A transfer instrument includes an outer cylinder and a carrier member (first carrier member) including a shaft (first shaft) and a support portion (first support portion) having a sheet shape. The support portion is capable of holding a medical sheet larger than the support portion in a width direction orthogonal to an axial direction of the outer cylinder. The support portion is capable of holding the medical sheet so as to bring a portion of the medical sheet protruding from the support portion into contact with another portion of the medical sheet with the support portion retracted in the outer cylinder and deformed into a curved shape.

### CROSS-REFERENCES TO RELATED APPLICATIONS

This application is based on and claims priority to Japanese Patent Application No. 2023-100675 filed on Jun. 20, 2023, the entire content of which is incorporated herein by reference.

### TECHNOLOGICAL FIELD

The present invention generally relates to a transfer instrument and a method for operating the same.

### BACKGROUND DISCUSSION

Japanese Patent Application Publication No. 2009-000511 (JP 2009-000511 A) discloses a transfer instrument for transferring a medical sheet (cell sheet) for use in, for example, organ transplantation to a treatment site of a living body. The transfer instrument includes an outer cylinder, a slide member slidably supported in the outer cylinder, and a seat support member provided at a distal end of the slide member.

### SUMMARY

It is desired to transfer a medical sheet to a treatment site more efficiently.

(1) A transfer instrument used to transfer a medical sheet to a treatment site of a living body includes an outer cylinder and a carrier member including a shaft and a support portion. The shaft extends in an extending direction and the support portion is positioned at a distal end of the shaft so that the support portion and the shaft move together. The shaft is positionable in the outer cylinder so that the shaft extends in an axial direction of the outer cylinder and is movable relative to the outer cylinder in the axial direction. The support portion has a sheet shape and includes a support surface configured to hold the medical sheet having a size larger than the support portion in a width direction orthogonal to the extending direction of the shaft so that a portion of the medical sheet protrude outwardly beyond the support portion. The shaft is movable in a proximal direction relative to the outer cylinder when the shaft is positioned in the outer cylinder and the support portion is in an extended position extending distally beyond the distal-end opening of the outer cylinder with the medical sheet overlying the support surface of the support portion to retract the support portion into the outer cylinder and cause the support portion to deform into a curved shape while also bringing a part of the protruding portion of the medical sheet into contact with another part of the medical sheet when the support portion is in a retracted position within the outer cylinder. The shaft is movable in a distal direction relative to the outer cylinder when the support portion is in the retracted position inside the outer cylinder to cause the support portion to move to the extended position in which the support portion extends distally beyond the distal-end opening of the outer cylinder.

With this configuration, the support portion can be retracted in the outer cylinder with the support portion deformed into a curved shape. It is therefore possible to reduce the diameter of the device (transfer instrument) and achieve less invasion. Further, since the medical sheet larger in width than the support portion can be retracted in the outer cylinder, it is possible to reduce the diameter of the device and achieve less invasion more effectively. Therefore, this transfer instrument can efficiently transfer the medical sheet to the treatment site. Further, since different portions of the medical sheet come into contact with each other with the medical sheet retracted the outer cylinder, it is easy to appropriately keep moisture of the medical sheet.

(2) The transfer instrument according to the above (1), in which the support portion may be configured to hold the medical sheet so as to bring portions of the medical sheet protruding from both sides of the support portion in the width direction into contact with each other with the support portion retracted in the outer cylinder and deformed into a curved shape. With this configuration, it is possible to reduce the diameter of the device and achieve less invasion more effectively.

(3) The transfer instrument according to the above (2), in which the support portion may be capable of holding the medical sheet so as to bring the portions of the medical sheet protruding from both the sides of the support portion in the width direction into contact with a portion of the medical sheet supported by the support surface with the support portion retracted in the outer cylinder and deformed into a curved shape. With this configuration, it is possible to reduce the diameter of the device and achieve less invasion more effectively.

(4) The transfer instrument according to any one of the above (1) to (3), in which the support portion may have a corrugated shape in cross section along the width direction of the support portion with the support portion unfolded by protruding from the distal-end opening of the outer cylinder, and with the support portion retracted in the outer cylinder and deformed into a curved shape, at least a part of the corrugated shape of the support portion may be curved to bulge toward the inside of the outer cylinder. The support portion configured as described above can hold the medical sheet having a larger width.

(5) A second aspect is a method comprising: moving a support portion toward a dish on which is located a medical sheet, with the support portion being fixed to a distal end portion of a shaft and the shaft being positioned in an outer cylinder so that a proximal end of the shaft extends proximally beyond a proximal end of the outer cylinder and the support portion is in an extended position in which the support portion extends distally beyond a distal-open end at a distal end of the outer cylinder, and wherein the support portion includes a support surface and an oppositely facing back surface. The method also involves: placing the medical sheet on the support surface while the support portion is in the extended position, with the placing of the medical sheet on the support surface including placing the medical sheet on the support surface such that a portion of the medical sheet extends outwardly beyond an outer periphery of the support portion; and retracting the support portion together with the medical sheet into the outer cylinder by moving the support portion relative to the outer cylinder from the extended position to a retracted position in which the support portion and the medical sheet are located inside the outer cylinder. During the retracting of the support portion and the medical sheet into the outer cylinder, the support portion is deformed into a curved shape and a part of the portion of the medical sheet extending outwardly beyond the outer periphery of the support portion contacting another portion of the medical sheet.

According to another aspect, a transfer instrument for transferring a medical sheet to a treatment site of a living body comprises: an outer cylinder having a through hole that extends from a distal-end opening at a distal end of the outer cylinder to a proximal-end opening at a proximal end of the outer cylinder; and a carrier member that includes a shaft and a support portion, with the shaft having a distal end portion and the support portion having a proximal-end support portion and a distal-end support portion. The proximal-end support portion terminates at a connection joint that is fixed to the distal end portion of the shaft so that movement of the shaft results in movement of the support portion. The shaft is positionable in the through hole in the outer cylinder and is axially movable in an axial direction relative to the outer cylinder to move the support portion from an extended position in which the support portion is distal of the distal-end opening of the outer cylinder with the medical sheet supported on the support portion to a retracted position in which the support portion and the medical sheet are retracted into the through hole in the outer cylinder by axially moving the shaft in a proximal direction relative to the outer cylinder, and being axially movable from the retracted position to the extended position by moving the shaft in a distal direction relative to the outer cylinder. The support portion has a width orthogonal to the axial direction, with the width of the support being greater than the distal-end opening at the distal end of the outer cylinder so that as the support portion is moved from the extended position to the retracted position the support portion contacts the distal end of the outer cylinder and is deformed into a curved shape together with the medical sheet.

Advantageously, the support portion can be retracted in the outer cylinder with the support portion deformed into a curved shape. It is therefore possible to reduce the diameter of the device (transfer instrument) and achieve less invasion. Further, since the medical sheet larger in width than the support portion can be retracted in the outer cylinder, it is possible to reduce the diameter of the device and achieve less invasion more effectively. Therefore, the transfer instrument can efficiently transfer the medical sheet to the treatment site. Further, since different portions of the medical sheet come into contact with each other with the medical sheet retracted the outer cylinder, it is easy to appropriately keep moisture of the medical sheet.

### BRIEF DESCRIPTION OF THE DRAWINGS

FIG. 1 is a perspective view of a transfer instrument according to an embodiment of the present invention.

FIG. 2 is an exploded perspective view of the transfer instrument illustrated in FIG. 1.

FIG. 3 is a plan view of a distal end of the transfer instrument illustrated in FIG. 1.

FIG. 4 is a longitudinal cross-sectional view taken along line IV-IV in FIG. 3.

FIG. 5 is a transverse cross-sectional view taken along line V-V in FIG. 3.

FIG. 6 is a flowchart illustrating a procedure of a transfer method for transferring a medical sheet using the transfer instrument illustrated in FIG. 1.

FIG. 7 is an explanatory diagram of a sheet placing process.

FIG. 8 is an explanatory diagram of a retracting process.

FIG. 9 is a transverse cross-sectional view taken along line IX-IX in FIG. 8.

FIG. 10 is an explanatory diagram of a positioning process.

FIG. 11 is an explanatory diagram of an unfolding process.

FIG. 12 is an explanatory diagram of a moving process.

FIG. 13 is an explanatory diagram of a withdrawing process.

FIG. 14A is a plan view of a first support portion according to a modification;

FIG. 14B is a cross-sectional view of the first support portion along a width direction.

FIG. 15 is a cross-sectional view of a state where the first support portion according to the modification is retracted in an outer cylinder together with a second support portion and a medical sheet.

### DETAILED DESCRIPTION

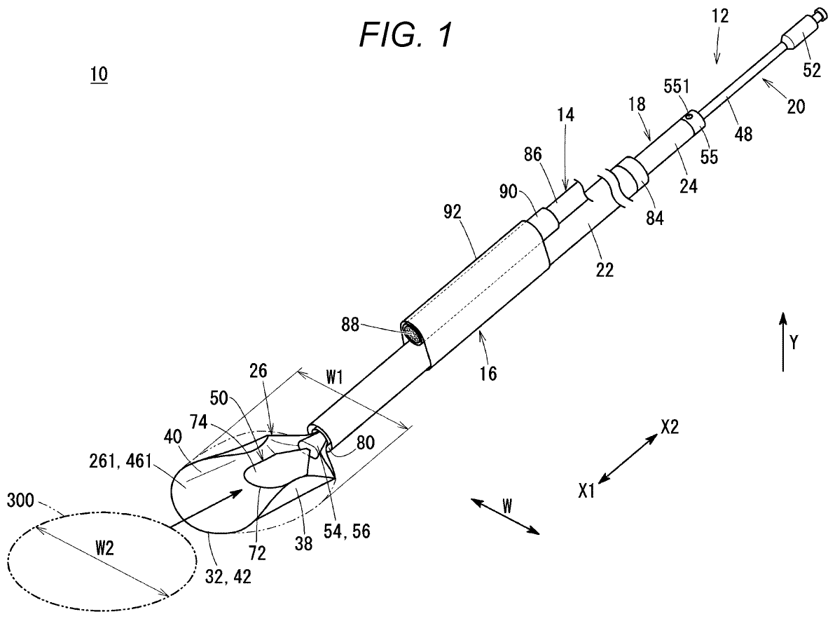

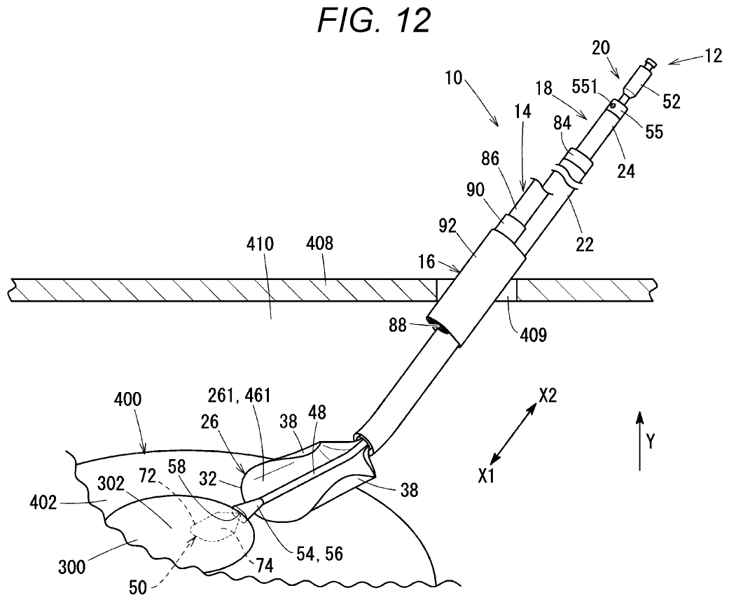

As illustrated in FIG. 1, a transfer instrument 10 according to the present embodiment is a medical instrument for transferring a medical sheet 300 to a treatment site of a living body. The transfer instrument 10 is used for, for example, the treatment of severe heart failure caused by ischemic heart disease. In this case, the medical sheet 300 is transplanted to a recipient site 402 of a heart 400 (the treatment site of the living body) (see FIG. 12). The transfer instrument 10 is capable of attaching a plurality of the medical sheets 300 to the recipient site 402.

Examples of such a medical sheet 300 include pharmaceutical products or regenerative medicine products for medical use, a medical instrument, and the like. The medical sheet 300 is formed in a sheet shape such as a film shape or a membrane shape (gel object). Fibrin or the like may be applied to the medical sheet 300 for reinforcement. Examples of the regenerative medicine products including cells include a cell sheet (sheet-shaped cell culture), a spheroid, and the like. It is possible to form the cell sheet by culturing autologous cells or allogenic cells. The cells constituting the cell sheet include, for example, somatic stem cells (adult stem cells), mesenchymal stem cells, or iPS cells (induced pluripotent stem cells)-derived cardiomyocytes. Examples of the somatic stem cells preferably include skeletal myoblast cells (myoblast cells).

The medical sheet 300 may contain a tissue adhesive, a local anesthetic, or the like. The medical sheet 300 has a thickness of, for example, about 100 μm, and has a diameter of, for example, about 60 mm. The thickness and the diameter (size) of the medical sheet 300 are not limited these values and can be set as desired.

The medical sheet 300 may be a sheet to be transplanted to an organ (for example, lung, liver, pancreas, kidney, small intestine, esophagus, or the like) other than the heart 400. Further, the medical sheet 300 may be, for example, an anti-adhesion sheet as long as the sheet is for medical use.

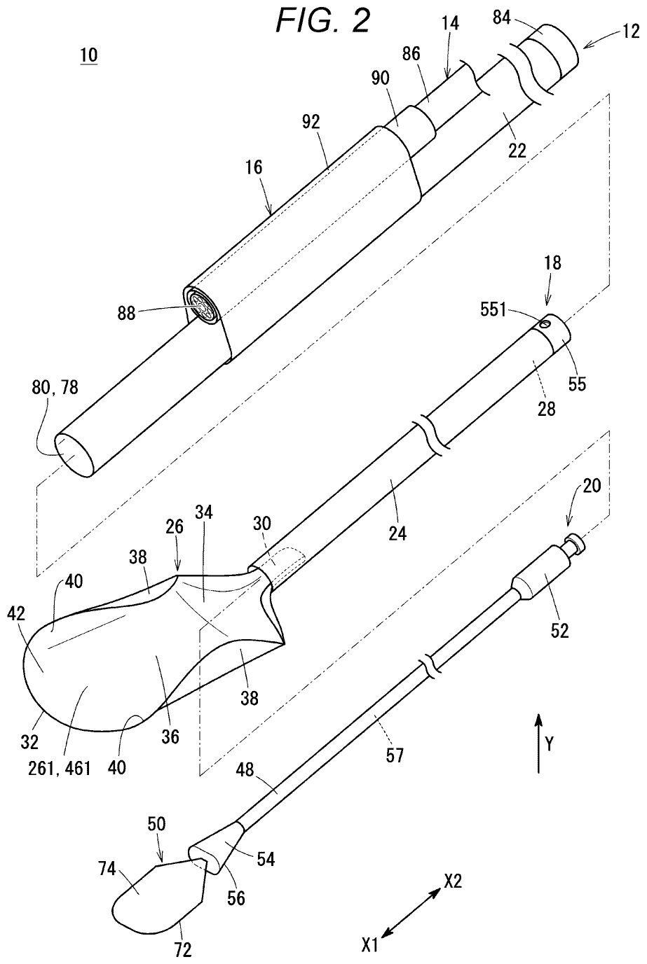

As illustrated in FIGS. 1 and 2, the transfer instrument 10 includes an instrument body 12, an endoscope 14, and a fixing member 16. The instrument body 12 includes a first carrier member 18, a second carrier member 20, and an outer cylinder 22. The transfer instrument 10 is not limited to a configuration including the endoscope 14.

In FIG. 2, the first carrier member 18 includes a first shaft 24 and a first support portion 26.

The first shaft 24 is a tubular body (in the present embodiment, a circular tube member) having a first lumen 28. The first lumen 28 opens at a distal end (end in a direction of arrow X1) of the first shaft 24 and opens at a proximal end (end in a direction of arrow X2) of the first shaft 24. The first shaft 24 has an airtight valve 55 provided at its proximal end with the valve 55 in close contact with an outer circumferential surface of the second shaft 48. The valve 55 has a marker 551 provided on its outer circumferential surface. When the transfer instrument 10 is used, the marker 551 is visible to the user. The first shaft 24 is not limited to a tubular body and may be a body other than a tubular body.

The first shaft 24 extends in an axial direction of the outer cylinder 22 and is disposed inside the outer cylinder 22 in a manner as to be movable along the axial direction. The first shaft 24 includes, for example, a resin material. Examples of the constituent material from which the first shaft 24 may be fabricated include, but are not particularly limited to, polyethylene, polypropylene, fluororesin, polyethylene terephthalate, polymethyl methacrylate, a polyamide resin, polystyrene, polycarbonate, polyimide, polyetherimide, polyetheretherketone, polyvinyl chloride, an ABS resin, a polyamide elastomer, and a polyester elastomer. The first shaft 24 may include a metal material.

The first shaft 24 may be flexible. The first shaft 24 may have a flexible tube portion capable of maintaining a bent shape. In this case, the first shaft 24 can be bent into an any desired shape in a body cavity and can maintain the bent shape. As illustrated in FIGS. 2 to 4, the first support portion 26 is attached to the distal end of the first shaft 24. The first support portion 26 includes or is fabricated from, for example, a resin material. The first support portion 26 can hold the medical sheet 300. A flexible resin sheet member (film member) is bent into a predetermined form to form the first support portion 26. Alternatively, the sheet member is shaped into a predetermined form by a sheet forming die to form the first support portion 26. It is preferable that the sheet member have, but not particularly limited to, a thickness of, for example, 100 μm or more and 200 μm or less. The first support portion 26 includes a first joint 30 and a first support body 32.

The constituent material from which the first support portion 26 may be fabricated preferably has transparency, and examples of the constituent material include, but not particularly limited to, polyethylene, polycarbonate, polyamide, polystyrene, polypropylene, polyacetal resin, polyimide, polyetherimide, polyetheretherketone, polyethylene terephthalate, and fluororesin. Further, the first support portion 26 may have a mesh shape.

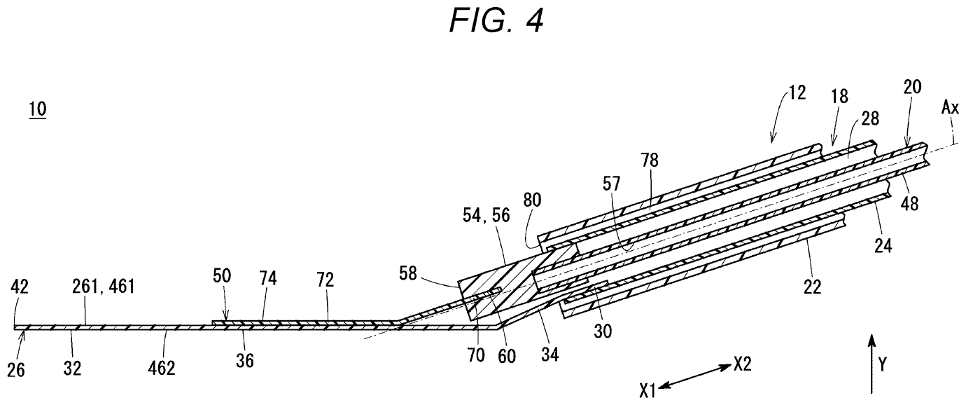

In FIG. 4, the first joint 30 is bonded to an inner circumferential surface of the distal end of the first shaft 24 with an adhesive. Examples of the adhesive include, but not particularly limited to, a UV adhesive, a hot-melt adhesive, and an instant adhesive (for example, cyanoacrylate-based instant adhesive). The first joint 30 may be thermally fused to the inner circumferential surface of the first shaft 24. The first support portion 26 may be detachable from the distal end of the first shaft 24.

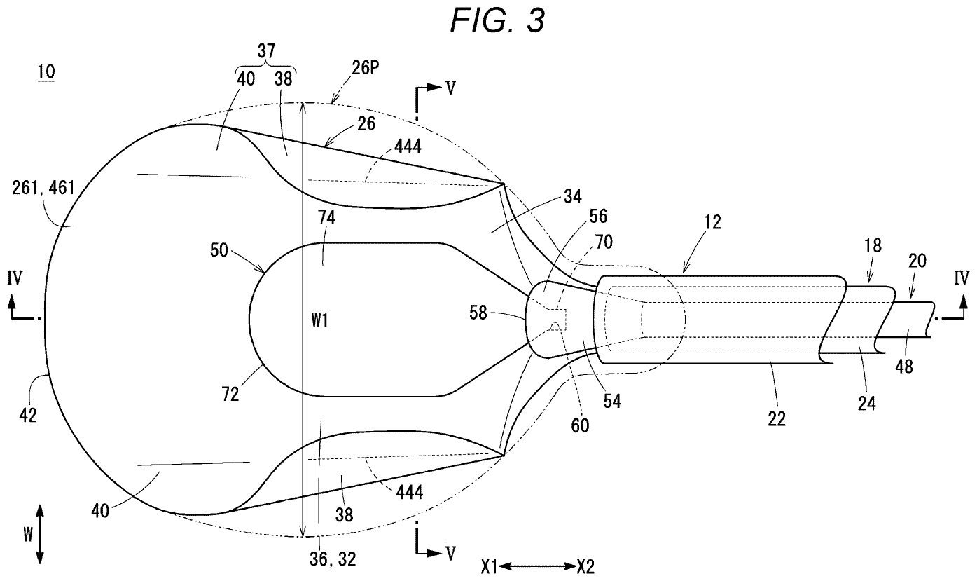

As illustrated in FIG. 2, the first support body 32 extends in a distal end direction from first joint 30. The first support body 32 includes a proximal-end support portion 34, an intermediate support portion 36, a pair of first protrusions 38, a pair of second protrusions 40, and a distal-end support portion 42. The first support body 32 includes a first support surface 261. The marker 551 of the first shaft 24 is disposed so as to face upward when the first support surface 261 faces upward. That is, the marker 551 allows the user to confirm the direction of the first support surface 261 of the first support portion 26. As shown in FIG. 3, the support portion 26 generally has a lengthwise extent between the distal-end support portion 42 (distal end portion) of the support portion 26 and the proximal-end support portion 34 (proximal end portion) of the support portion 26. Also, as seen in a plan view (FIG. 3), an imaginary continuation of the central axis of the first shaft 24 passes through the proximal-end support portion 34 of the first support body 32 and the distal-end support portion 42 of the first support body 32.

As illustrated in FIG. 3, the proximal-end support portion 34 is formed to be wider in its extending direction. In other words, both sides of the proximal-end support portion 34 in a width direction are tapered toward the first joint 30 (see FIG. 2). The intermediate support portion 36 is formed in a tapered shape so as to be gradually narrower in width from the distal end toward the proximal end (in the direction of arrow X2).

The distal-end support portion 42 is connected to a distal end of the intermediate support portion 36 and distal ends of the pair of second protrusions 40. The distal-end support portion 42 protrudes in an arc shape in the distal end direction (the direction of arrow X1). That is, when viewed from a direction perpendicular to the first support surface 261 illustrated in FIG. 3, the distal-end support portion 42 of the first support portion 26 has an arc shape connecting the pair of second protrusions 40.

As illustrated in FIG. 4, the proximal-end support portion 34 extends roughly along an axis Ax of the first shaft 24 from a distal end of the first joint 30 in the distal end direction (the direction of arrow X1). The intermediate support portion 36 intersects with the axis Ax of the first shaft 24 and extends from a distal end of the proximal-end support portion 34 toward the distal end of the first support portion 26 (in the direction of arrow X1).

In FIGS. 2 and 3, the pair of first protrusions 38 protrude upward (in the direction of arrow Y) from both sides of the intermediate support portion 36 in the width direction orthogonal to a direction in which the first shaft 24 moves and inward in the width direction of the intermediate support portion 36. The pair of first protrusions 38 are connected to the proximal-end support portion 34. Each first protrusion 38 has a convex shape extending in a direction away from the first support surface 261.

As illustrated in FIG. 3, the pair of second protrusions 40 are connected to distal ends of the pair of first protrusions 38. The pair of second protrusions 40 protrude upward from both sides of the intermediate support portion 36 in the width direction and outward in the width direction of the intermediate support portion 36. Each second protrusion 40 is formed with a smaller curvature than the first protrusion 38. The second protrusions 40 are lower in protrusion height relative to the first support surface 261 than the first protrusions 38.

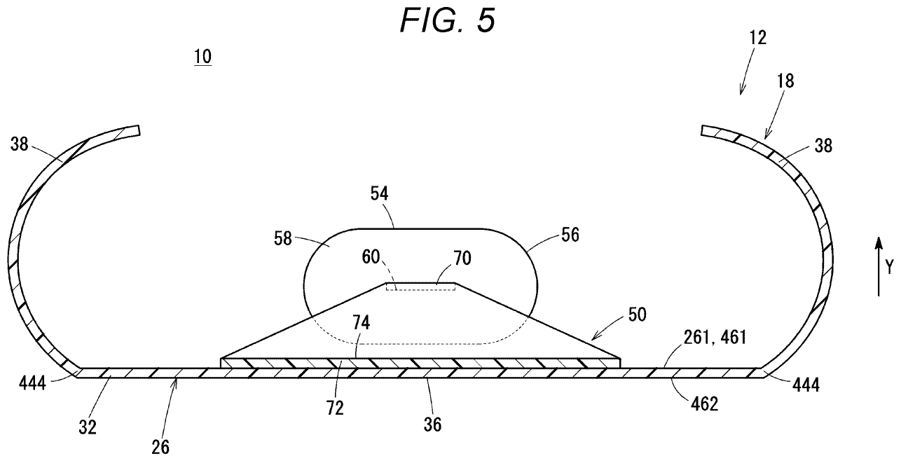

Each of the pair of first protrusions 38 has one of a pair of bent portions 444. Each of the pair of bent portions 444 causes a corresponding one of the pair of first protrusions 38 to bend from the first support surface 261 (intermediate support portion 36) of the first support portion 26 (see FIG. 5).

The first support body 32 includes a front surface 461 that faces upward (in the direction of arrow Y) and includes the first support surface 261, and a back surface 462 that is a surface opposite to the front surface 461. The first support surface 261 includes a flat surface continuous over an upper surface of the proximal-end support portion 34 and upper surfaces of the intermediate support portion 36 and the distal-end support portion 42. A lubricant may be applied to the first support surface 261 so as to allow a second support portion 50 (to be described later) of the second carrier member 20 to smoothly slide on the first support surface 261.

As illustrated in FIG. 2, the second carrier member 20 includes a second shaft 48, the second support portion 50, and a hub 52.

The second shaft 48 is a tubular body (in the present embodiment, a circular tube member) having a second lumen 57. The second shaft 48 is longer in the axial direction than the first shaft 24. The second shaft 48 is inserted into the first lumen 28 of the first shaft 24 (see FIGS. 1 and 4). In other words, a distal end of the second shaft 48 protrudes in the distal end direction (the direction of arrow X1) relative to the distal-end opening of the first shaft 24. A proximal end of the second shaft 48 protrudes in the proximal end direction (the direction of arrow X2) relative to the proximal-end opening of the first shaft 24 (see FIG. 1). The second shaft 48 is provided extending along the first shaft 24 so as to be movable along the first shaft 24. The second shaft 48 is not limited to a tubular body and may be a body other than a tubular body.

The second shaft 48 is configured to follow the shape of the first support portion 26. As the constituent material from which the second shaft 48 may be fabricated, for example, a material more flexible than the constituent material of the first shaft 24 is selected. Specifically, examples of the constituent material of the second shaft 48 include a polyamide elastomer, a polyester elastomer, a polyurethane elastomer, polyvinyl chloride, polybutadiene, a silicone rubber, and a metal coil (including a composite with a resin). The second shaft 48 is flexible.

As illustrated in FIG. 4, the second shaft 48 includes a carrier holding portion 54 and a pressure-application portion 56 that is the distal end of the second shaft 48. The pressure-application portion 56 includes an elastic body such as an elastomer member. The pressure-application portion 56 presses the first support portion 26 against an inner surface of the outer cylinder 22 with the first support portion 26 retracted in the outer cylinder 22.

A distal end of the carrier holding portion 54 includes a pressing surface 58. The carrier holding portion 54 can cause the pressing surface 58 to press an outer edge surface of the medical sheet 300 supported by the first support portion 26 in the distal end direction (the direction of arrow X1). In the present embodiment, the pressure-application portion 56 is provided with the carrier holding portion 54 that supports the second support portion 50. The carrier holding portion 54 includes the pressing surface 58 and an attachment hole 60.

In FIG. 4, the pressing surface 58 is provided on a distal end surface of the carrier holding portion 54. The attachment hole 60 opens at the pressing surface 58. The second support portion 50 is attached to the pressing surface 58. The pressing surface 58 presses the outer edge surface of the medical sheet 300 in the distal end direction (the direction of arrow X1) (see FIG. 12).

In FIGS. 2 to 4, the second support portion 50 is a flexible sheet. The second support portion 50 includes a second joint 70 and a second support body 72. The second joint 70 is provided at a proximal end of the second support portion 50. The second joint 70 is provided at a proximal end of the second support body 72. The second joint 70 is inserted into or positioned in the attachment hole 60 of the carrier holding portion 54, and is bonded, for example. The second joint 70 may be joined to the attachment hole 60 of the carrier holding portion 54 by a suitable joining method other than adhesion. The second support portion 50 may be integrally formed with the carrier holding portion 54.

The second support body 72 extends in the distal end direction (the direction of arrow X1) from the second joint 70. The second support body 72 extending from the second joint 70 is shorter in the extending direction than the first support body 32 extending from the first joint 30. The second support body 72 is provided, on its upper surface, with a second support surface 74 on which the medical sheet 300 is placed. The second support surface 74 includes a flat surface. The second support body 72 is smaller than the first support body 32. That is, the second support surface 74 is smaller in area than the first support surface 261.

In FIG. 2, the hub 52 is attached to the proximal end of the second shaft 48.

In FIGS. 1 and 2, the outer cylinder 22 is a cylindrical member having a lumen 78. The lumen 78 has a distal-end opening 80 that opens at a distal end (end in the direction of arrow X1) of the outer cylinder 22. The lumen 78 opens at a proximal end (end in the direction of arrow X2) of the outer cylinder 22. The outer cylinder 22 is flexible. Examples of the constituent material from which the outer cylinder 22 may be fabricated are the same as the examples of the constituent material of the first shaft 24 described above. The first shaft 24 is inserted into or positioned in the lumen 78 of the outer cylinder 22. The outer cylinder 22 is shorter in length along the axial direction than the first shaft 24. The outer cylinder 22 is provided with, at its proximal end, an airtight valve 84 that is in close contact with an outer circumferential surface of the first shaft 24.

In FIGS. 2 and 4, a distal end surface of the outer cylinder 22 extends orthogonal to the axial direction of the outer cylinder 22.

As illustrated in FIG. 2, the endoscope 14 includes a long or elongated endoscope main body 86. A distal end of the endoscope main body 86 is fixed to an outer circumferential surface of the outer cylinder 22 by the fixing member 16 (see FIG. 1). An objective lens 88 provided on a distal end surface of the endoscope main body 86 is oriented toward the distal end of the outer cylinder 22 (in the direction of arrow X1). The distal end of the endoscope main body 86 is fixed to an intermediate portion of the outer cylinder 22 in the axial direction. The distal end of the endoscope main body 86 may be fixed to the distal end of the outer cylinder 22.

The fixing member 16 includes, for example, a fixing cylinder 90 and a fixing tube 92. The fixing cylinder 90 includes, for example, a hard resin material. The endoscope main body 86 can be inserted into or positioned in a lumen of the fixing cylinder 90. The fixing cylinder 90 is disposed along a longitudinal direction of the outer cylinder 22. The fixing tube 92 is a tube for fixing the fixing cylinder 90 at a predetermined position of the outer cylinder 22. The fixing tube 92 is, for example, a heat-shrink tube. The manner in which the distal end of the endoscope main body 86 is fixed to the outer cylinder 22 may be determined as desired.

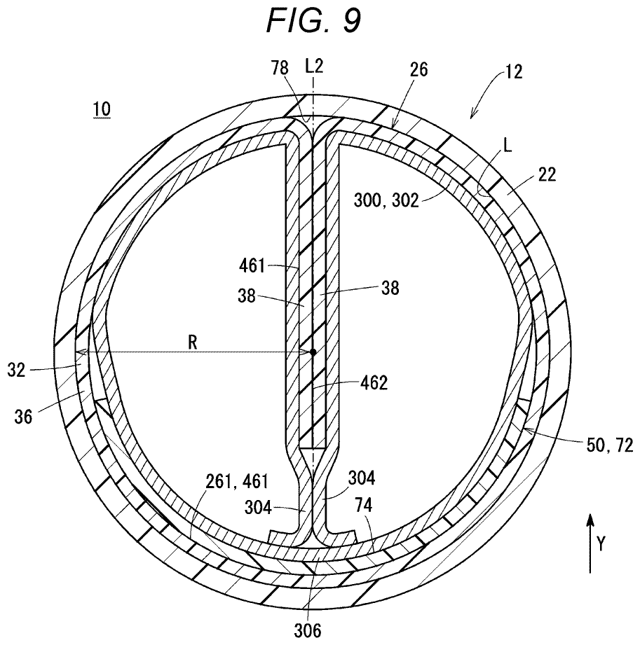

As illustrated in FIG. 3, a portion including both the first protrusion 38 and the second protrusion 40 of the first support portion 26 is hereinafter referred to as “protrusion 37”. Therefore, the protrusion 37 is provided as a pair on both sides of the first support portion 26 in the width direction. In FIG. 3, the direction (W direction) orthogonal to the axial direction of the first shaft 24 refers to the width direction of the first support portion 26. The W direction also refers to a width direction of other components (the outer cylinder 22 and the like). In the present embodiment, the first support portion 26 includes the pair of protrusions 37, and W1 in FIG. 3 denotes a width of the widest portion of the first support portion 26 when the first support portion 26 is unfolded to be flat. That is, the width W1 of the first support portion 26 (the width W1 orthogonal to an imaginary continuation of the central axis of the first shaft 24) is the maximum width of a first support portion 26P in a flat state before the pair of protrusions 37 are formed (before folded). The width W1 of the first support portion 26 is larger than an inner circumferential length L of the outer cylinder 22 (see FIG. 9). As shown in FIGS. 1, 3 and 7, the width W1 of the first support portion 26 is greater than the distal-end opening 80 of the outer cylinder 22. In FIG. 9, the inner circumferential length L of the outer cylinder 22 is a length of an inner circumferential surface along a circumferential direction of the outer cylinder 22 (L=2πR). R denotes the radius of the lumen 78 of the outer cylinder 22. As illustrated in FIG. 1, the first support portion 26 can hold the medical sheet 300 larger than the first support portion 26 in the width direction (W direction) orthogonal to the axial direction of the outer cylinder 22. A width W2 of the medical sheet 300 described in the present embodiment is larger than the width W1 of the first support portion 26. In a case where the medical sheet 300 is circular, the width W2 is the diameter of the medical sheet 300.

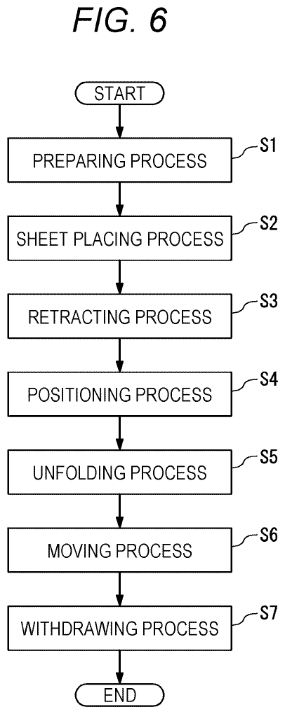

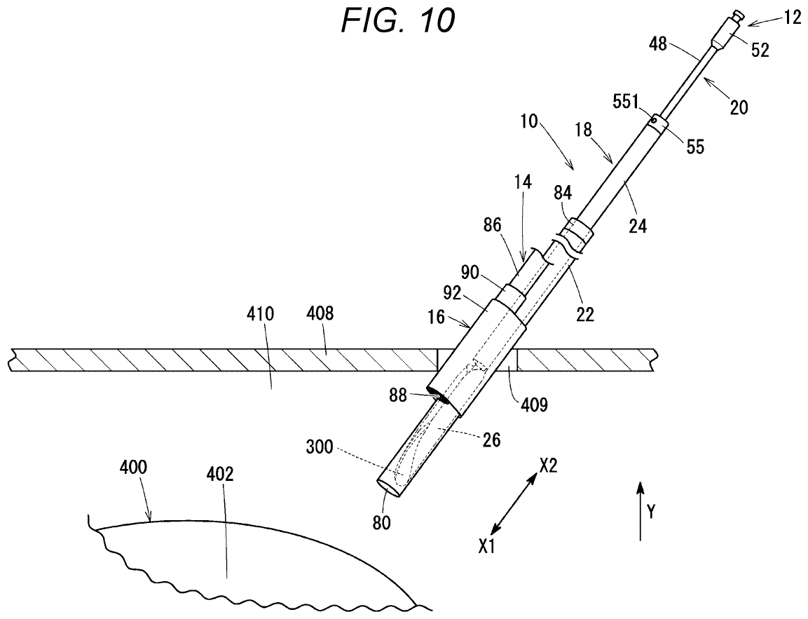

Next, a transfer method for transferring the medical sheet 300 to a treatment site of a living body will be described. Specifically, as illustrated in FIGS. 10 to 13, the transfer method for transferring the medical sheet 300 to the recipient site 402 of the heart 400 (the treatment site of the living body) during thoracoscopic surgery will be described. As illustrated in FIG. 6, the transfer method according to the present embodiment includes a preparing process, a sheet placing process, a retracting process, a positioning process, an unfolding process, a moving process, and a withdrawing process.

First, in the preparing process (step S1), the transfer instrument 10 according to the present embodiment described above is prepared. The following description will be given on the assumption that a state as illustrated in FIG. 1 is an initial state of the transfer instrument 10. In the initial state, the first shaft 24 and the second shaft 48 are moved in the distal end direction (the direction of arrow X1) relative to the outer cylinder 22 so as to be at a protruding position (second position) where the first support portion 26 and the second support portion 50 protrude from the distal-end opening 80 of the outer cylinder 22 in the distal end direction. The first and second support portions 26 and 50 are each unfolded when moving out of the outer cylinder 22 in the distal end direction, and the second support portion 50 is positioned on the first support surface 261 of the first support portion 26. That is, the second support portion 50 is positioned at a retracted position where the second support portion 50 is placed on the first support surface 261 of the first support portion 26. At this time, a proximal end of the carrier holding portion 54 is in the first lumen 28 of the first shaft 24.

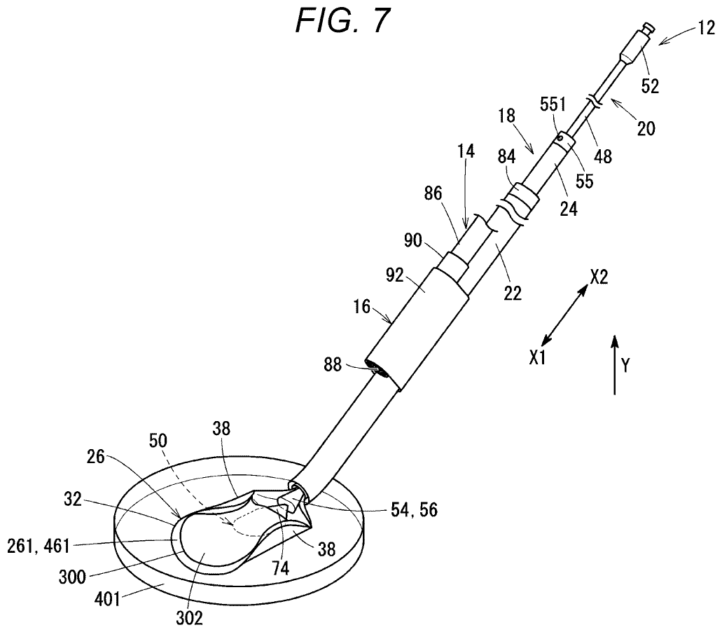

Next, in the sheet placing process (step S2), as illustrated in FIG. 7, the medical sheet 300 placed on or located in a Petri dish 401 is placed on the second support surface 74. The medical sheet 300 protrudes outward from the second support portion 50 with the medical sheet 300 placed on the second support surface 74. The first support surface 261 supports an overhanging portion 302 of the medical sheet 300 that sticks out over the second support portion 50. Since the width W2 of the medical sheet 300 is larger than the width W1 of the first support portion 26 (see FIG. 1), as illustrated in FIG. 7, with the medical sheet 300 placed on the first support portion 26, both ends of the medical sheet 300 in the width direction protrude from both ends of the first support portion 26.

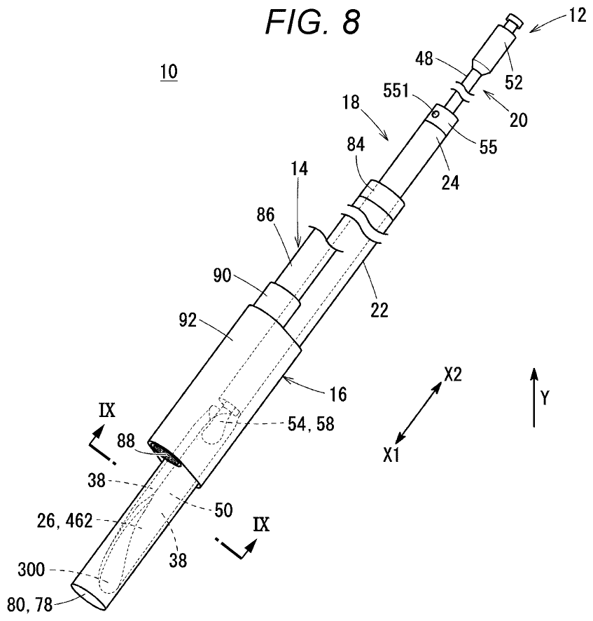

Subsequently, in the retracting process (step S3 in FIG. 6), as illustrated in FIG. 8, the medical sheet 300 is retracted in the outer cylinder 22 together with the first support portion 26 and the second support portion 50 at a retracted position (first position). Specifically, the first shaft 24 of the first carrier member 18 and the second shaft 48 of the second carrier member 20 are moved together in the proximal end direction (the direction of arrow X2) relative to the outer cylinder 22.

Then, the proximal-end support portion 34 (see FIG. 2) of the first support portion 26 is pulled in the proximal end direction through the distal-end opening 80 of the outer cylinder 22. At this time, when both the tapered sides of the proximal-end support portion 34 come into contact with the distal-end opening 80 of the outer cylinder 22, a force acts on the proximal-end support portion 34 to cause the proximal-end support portion 34 to curl along the circumferential direction of the outer cylinder 22. Therefore, the proximal-end support portion 34 is smoothly pulled into the outer cylinder 22 while curling. At this time, the first support portion 26 is retracted in the outer cylinder 22 while curling in a conical shape such that the distal end side of the first support portion 26 becomes larger in diameter than the proximal-end support portion 34.

When the proximal-end support portion 34 of the first support portion 26 becomes deformed, a force acts on the intermediate support portion 36 of the first support portion 26 to cause the intermediate support portion 36 to curl along the circumferential direction of the outer cylinder 22, so that the intermediate support portion 36 (see FIG. 2) is pulled into the outer cylinder 22 while curling. At this time, the intermediate support portion 36 becomes deformed into a cylindrical shape along the inner surface of the outer cylinder 22. Each of the pair of first protrusions 38 is curved such that the front surface 461 of the first support portion 26 faces inward and the back surface 462 of the first support portion 26 faces outward. As illustrated in FIG. 9, the back surfaces 462 of both sides of the first support portion 26 in the width direction come into contact with each other on an imaginary line L2 extending orthogonal to the center axis of the outer cylinder 22. One of the first protrusions 38 and the other first protrusion 38 come into contact with each other and are retracted downward (toward the first support surface 261, the front surface 461).

Accordingly, the back surface 462 of the first support portion 26 is curved in close contact with the inner surface of the outer cylinder 22, each of the first protrusions 38 is further curved to fold back toward the center of the outer cylinder 22, and a free end of each of the first protrusions 38 is positioned below the center axis of the outer cylinder 22. That is, the first support portion 26 is curved in a heart shape (as seen with reference to the transverse cross-section of FIG. 9) along the inner surface of the outer cylinder 22.

The heart shape refers to a roughly round shape including two convex shapes, a convex shape on one side and a convex shape on the other side (i.e., a convex shape on one side of the imaginary line L2 and a convex shape on the other side of the imaginary line L2). In a case where the heart shape is formed in the lumen 78 of the tubular body (outer cylinder 22), the two convex shapes protruding toward the opposite sides along the inner surface of the tubular body come close to each other so as to bring their respective circumferential surfaces into partial contact with each other, so that the entire contour becomes a roughly round shape along the inner surface of the tubular body (refer to the shape of the first support portion 26 in FIG. 9).

Along with the curved deformation of the first support portion 26, the second support portion 50 is similarly curved and deformed along the first support portion 26 inside the first support portion 26 (on the front surface 461 side). Along with the curved deformation of the first support portion 26 and the second support portion 50, the medical sheet 300 is deformed into a shape corresponding to the shape of the first support body 32 and the shape of the second support body 72, and the medical sheet 300 is retracted in the outer cylinder 22 accordingly.

As illustrated in FIG. 9, with the first support portion 26 retracted in the outer cylinder 22 and deformed into a curved shape, a portion of the medical sheet 300 protruding from the first support portion 26 (i.e., a portion of the medical sheet 300 protruding outwardly beyond the first support portion 26) comes into contact with another portion of the medical sheet 300. In the example illustrated in FIG. 9, the medical sheet 300 has portions (hereinafter, also referred to as “protruding portions 304”) protruding from or extending outwardly beyond both sides of the first support portion 26 in the width direction, and the protruding portions 304 are in contact with each other. Further, the pair of protruding portions 304 are in contact with a portion (hereinafter, also referred to as “sheet intermediate portion 306”) of the medical sheet 300 supported by the first support surface 261.

With the medical sheet 300 placed on the first support portion 26, a center of the first support portion 26 in the width direction need not coincide with a center of the medical sheet 300 in the width direction. That is, the center of the medical sheet 300 in the width direction may be shifted in the width direction (left-right direction) relative to the center of the first support portion 26 in the width direction. Only one of the protruding portions 304 of the medical sheet 300 may be in contact with the sheet intermediate portion 306. Neither of the pair of protruding portions 304 of the medical sheet 300 may be in contact with the sheet intermediate portion 306.

A partition wall (not illustrated) protruding downward from an upper inner surface of the outer cylinder 22 may be provided inside the distal end of the outer cylinder 22. The back surfaces 462 of the pair of first protrusions 38 of the first support portion 26 may come contact with the partition wall. When the first support portion 26 and the medical sheet 300 are stored in the outer cylinder 22, the partition wall may function as a guide that causes the first support portion 26 to be curved and deformed into a heart shape.

As illustrated in FIG. 8, when the first support portion 26 is entirely inserted into or positioned in the outer cylinder 22, the retracting process is complete. At the completion of the retracting process, the first support portion 26 may partially protrude from the distal-end opening 80 of the outer cylinder 22. In this case, the state where the first support portion 26 partially protrudes from the distal-end opening 80 of the outer cylinder 22 corresponds to the first position of the first support portion 26.

Subsequently, in the positioning process (step S4 in FIG. 6), as illustrated in FIG. 10, the transfer instrument 10 is inserted into a chest cavity 410 through an incision 409 in a chest 408. At this time, the distal end of the transfer instrument 10 is positioned near the recipient site 402 of the heart 400, and the distal end of the endoscope 14 is positioned in the chest cavity 410. Before the transfer instrument 10 is inserted into the chest cavity 410, a liquid supply instrument (not illustrated) may be connected to a connection port of the hub 52 to introduce a liquid (for example, a physiological saline solution).

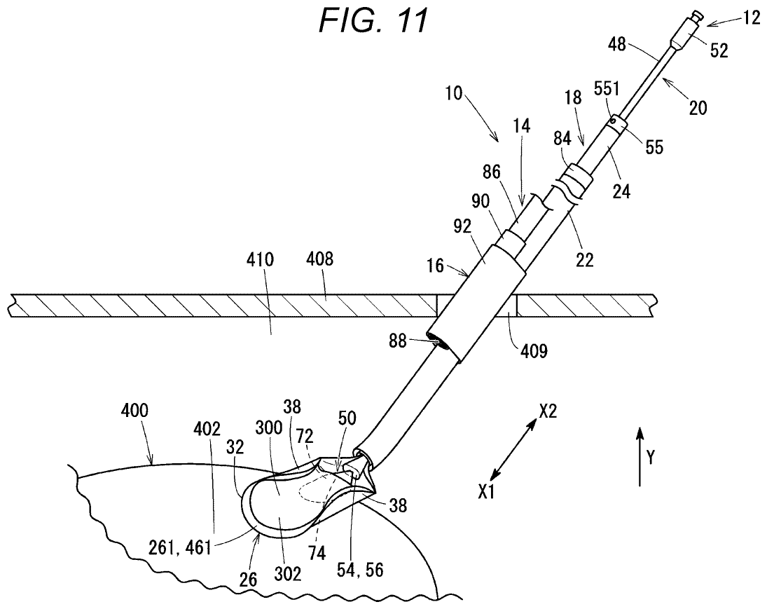

Subsequently, in the unfolding process (step S5 in FIG. 6), as illustrated in FIG. 11, the first support portion 26, the second support portion 50, and the medical sheet 300 are unfolded. Specifically, in the unfolding process, with the first shaft 24 gripped, the first shaft 24 is moved in the distal end direction (the direction of arrow X1) relative to the outer cylinder 22. Accordingly, the valve 55 of the first shaft 24 causes the second shaft 48 to move together with the first shaft 24 in the distal end direction (the direction of arrow X1). Then, the first support portion 26 that has moved out of the distal-end opening 80 of the outer cylinder 22 returns to the original shape due to its restoring force. At the second position where the first support portion 26 is unfolded, the second support portion 50 unrolls flat.

In the unfolding process, of the second carrier member 20, the second support surface 74 on which the medical sheet 300 is placed is entirely positioned on the first support surface 261. At this time, the medical sheet 300 is supported by the first support surface 261 and the second support surface 74. It is therefore possible to prevent the overhanging portion 302 of the medical sheet 300 from becoming crinkled before the medical sheet 300 is transferred to the recipient site 402 of the heart 400.

Next, in the moving process (step S6 in FIG. 6), as illustrated in FIG. 12, the second carrier member 20 is moved in the distal end direction (the direction of arrow X1) relative to the first carrier member 18, so that the second support portion 50 on which the medical sheet 300 is placed is moved from the retracted position to an advanced position, and the second support portion 50 protrudes in the distal end direction (the direction of arrow X1) relative to the distal end of the first support portion 26. Specifically, in the moving process, the second shaft 48 is moved in the distal end direction relative to the first shaft 24.

Accordingly, the second support portion 50 moves in the distal end direction (the direction of arrow X1) relative to the first support portion 26. At this time, when the distal end surface of the carrier holding portion 54 (pressure-application portion 56) presses the outer edge surface of the medical sheet 300 in the distal end direction, the medical sheet 300 is entirely positioned further than the first support portion 26 in the distal end direction. In this moving process, the medical sheet 300 is moved to above the recipient site 402 of the heart 400 to bring the overhanging portion 302 of the medical sheet 300 into contact with the recipient site 402.

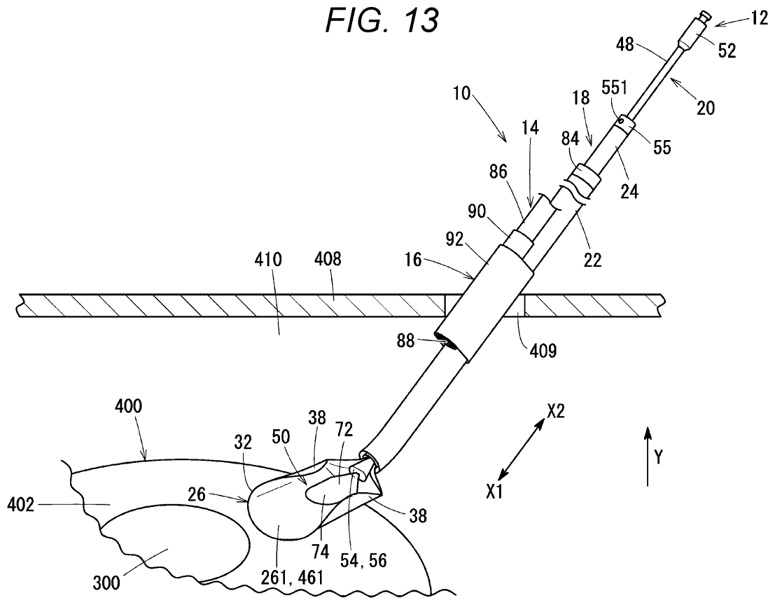

Subsequently, in the withdrawing process (step S7 in FIG. 6), as illustrated in FIG. 13, the second carrier member 20 is moved from the second position to the first position to withdraw the second support portion 50 from between the recipient site 402 and the medical sheet 300. Accordingly, the medical sheet 300 entirely comes into contact with the surface of the recipient site 402. This is the end of the transfer of the medical sheet 300 to the recipient site 402. Subsequently, the transfer instrument 10 is withdrawn from the chest 408 with the first support portion 26 and the second support portion 50 retracted in the outer cylinder 22.

The present embodiment has the following effects.

Since the transfer instrument 10 includes the second carrier member 20 movable in the axial direction relative to the first carrier member 18, it is possible to transfer the medical sheet 300 from above the first support portion 26 to the recipient site 402 of the living body using the second carrier member 20 without using another device (forceps or the like). It is therefore possible to efficiently transfer the medical sheet 300 to the recipient site 402.

As illustrated in FIG. 1, the second support portion 50 having a sheet shape and including the second support surface 74 capable of holding the medical sheet 300 is provided at the distal end of the second shaft 48. The second support portion 50 is movable relative to the first support portion 26 between the retracted position where the second support portion 50 is placed on the first support surface 261 and the advanced position located in the distal end direction (the direction of arrow X1) relative to the first support surface 261. With this configuration, the medical sheet 300 can be moved along the first support surface 261 of the first support portion 26 by the second support portion 50 including the second support surface 74.

As illustrated in FIG. 9, at the retracted position (first position), the second support portion 50 is retracted in the outer cylinder 22 together with the first support portion 26 with the second support portion 50 deformed into a curved shape. With this configuration, even the second support portion 50 having a width dimension larger than the width (thickness) of the second shaft 48 can be suitably retracted in the outer cylinder 22 together with the first support portion 26 in the retracting process.

The first support portion 26 is a flexible sheet having the front surface 461 and the back surface 462. The first support portion 26 includes the pair of first protrusions 38 protruding upward from both sides of the first support surface 261 in the width direction orthogonal to the direction in which the first shaft 24 moves. With this configuration, when the first support portion 26 is retracted in the outer cylinder 22, the back surfaces 462 of the pair of first protrusions 38 curved and deformed into a convex shape come into contact with each other. The pair of first protrusions 38 are moved toward the first support surface 261, and the first support portion 26 is curved and deformed into a heart shape. Accordingly, the first support portion 26 can be retracted with the heart shape in the outer cylinder 22, so that it is possible to effectively reduce the possibility of breakage of the medical sheet 300 held by the first support portion 26 as compared with a case where the first support portion 26 is deformed into a shape other than the heart shape. Further, since the first support portion 26 can be smoothly and compactly retracted in the outer cylinder 22, the diameter of the outer cylinder 22 can be reduced as compared with a configuration where the first support portion 26 is not curved and deformed into a heart shape.

With the first support portion 26 retracted in the outer cylinder 22 and deformed into a curved shape, the first support portion 26 can hold the medical sheet 300 so as to bring a portion (protruding portion 304) of the medical sheet 300 protruding from the first support portion 26 into contact with another portion of the medical sheet 300. With this configuration, the first support portion 26 can be retracted in the outer cylinder 22 with the first support portion 26 deformed into a curved shape. It is therefore possible to reduce the diameter of the device (transfer instrument 10) and achieve less invasion. Further, since the medical sheet 300 larger in width than the first support portion 26 can be retracted in the outer cylinder 22, it is possible to reduce the diameter of the device and achieve less invasion more effectively. Therefore, the transfer instrument 10 can efficiently transfer the medical sheet 300 to the recipient site 402 (treatment site). Further, since different portions of the medical sheet 300 come into contact with each other with the medical sheet 300 retracted the outer cylinder 22, it is easy to appropriately keep moisture of the medical sheet 300.

The first support portion 26 can hold the medical sheet 300 so as to bring portions (protruding portions 304) of the medical sheet 300 protruding from both sides of the first support portion 26 in the width direction into contact with each other with the first support portion 26 retracted in the outer cylinder 22 and deformed into a curved shape. With this configuration, it is possible to reduce the diameter of the device and achieve less invasion more effectively.

The first support portion 26 can hold the medical sheet 300 so as to bring portions (protruding portions 304) of the medical sheet 300 protruding from both sides of the support portion in the width direction into contact with a portion (sheet intermediate portion 306) of the medical sheet 300 supported by the first support surface 261 with the first support portion 26 retracted in the outer cylinder 22 and curved and deformed.

With this configuration, it is possible to reduce the diameter of the device and achieve less invasion more effectively.

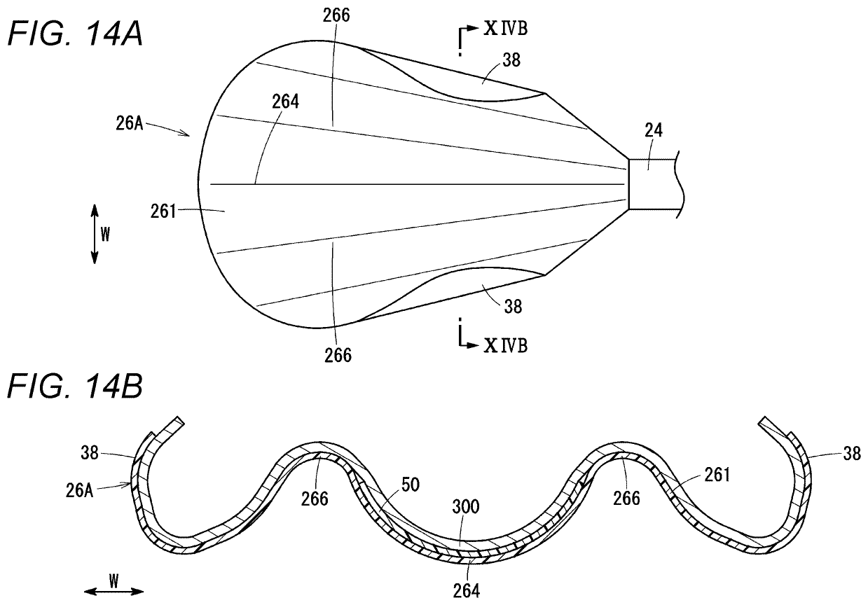

A first support portion 26A illustrated in FIGS. 14A and 14B may be adopted instead of the first support portion 26 described above. With the first support portion 26A unfolded by protruding from the distal-end opening 80 of the outer cylinder 22 (see FIG. 1), the first support portion 26A has a corrugated shape in cross section along the width direction (W direction) of the first support portion 26A. In FIG. 14A, the second support portion 50 and the medical sheet 300 are not illustrated. As illustrated in FIG. 14B, the first support portion 26A includes a downward curved convex portion 264 curved into a downward convex shape at the center in the width direction, and a pair of upward curved convex portions 266 curved into an upward convex shape on both sides of the downward curved convex portion 264. A pair of first protrusions 38 are further provided outside in the width direction of the pair of upward curved convex portions 266.

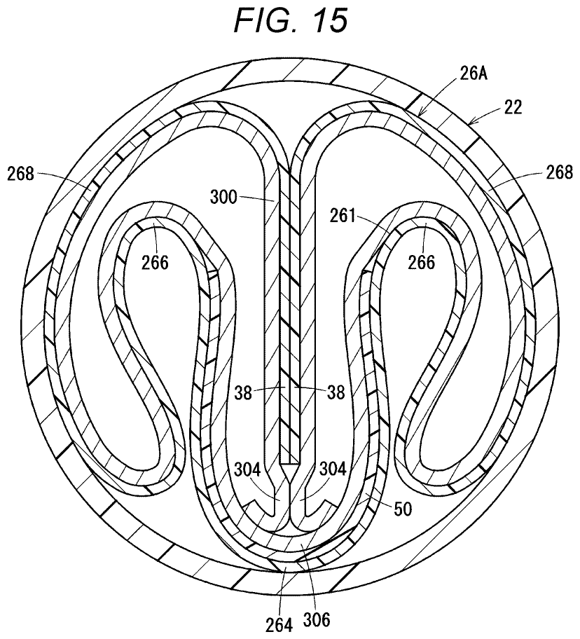

As illustrated in FIG. 15, with the first support portion 26A retracted in the outer cylinder 22 and deformed into a curved shape, at least a part of the corrugated shape of the first support portion 26A is curved to bulge toward the inside of the outer cylinder 22. In the example illustrated in FIG. 15, the pair of upward curved convex portions 266 are curved to bulge toward the inside of the outer cylinder 22 (space between a portion 268 of the first support portion 26A curved along the inner surface of the outer cylinder 22 and each of the first protrusions 38). The medical sheet 300 and the second support portion 50 are also curved along the curved shape of the first support portion 26A. The sheet intermediate portion 306 of the medical sheet 300 is supported by the downward curved convex portion 264 of the first support portion 26A and comes into contact with the pair of protruding portions 304 of the medical sheet 300.

Since the first support portion 26A configured as described above has a corrugated shape, it is possible to hold the medical sheet 300 having a larger width.

The present invention is not limited to the above-described disclosure, and various configurations can be adopted without departing from the gist of the present invention. That is, the detailed description above describes embodiments of a transfer instrument and transfer method for transferring a medical sheet to a treatment site of a living body, representing examples of the new transfer instrument and transfer method disclosed here, but the invention is not limited to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims

What is claimed is:

1. A transfer instrument used to transfer a medical sheet to a treatment site of a living body, the transfer instrument comprising:
an outer cylinder;
a carrier member including a shaft and a support portion, the shaft extending in an extending direction and the support portion being positioned at a distal end of the shaft so that the support portion and the shaft move together, the shaft being positionable in the outer cylinder so that the shaft extends in an axial direction of the outer cylinder and is movable relative to the outer cylinder in the axial direction, the support portion having a sheet shape and including a support surface configured to hold the medical sheet medical sheet having a size larger than the support portion in a width direction orthogonal to the extending direction of the shaft so that a portion of the medical sheet protrudes outwardly beyond the support portion;
the shaft being movable in a proximal direction relative to the outer cylinder when the shaft is positioned in the outer cylinder and the support portion is in an extended position extending distally beyond the distal-end opening of the outer cylinder with the medical sheet overlying the support surface of the support portion to retract the support portion into the outer cylinder and cause the support portion to deform into a curved shape while also bringing a part of the protruding portion of the medical sheet into contact with another part of the medical sheet when the support portion is in a retracted position within the outer cylinder; and
the shaft being movable in a distal direction relative to the outer cylinder when the support portion is in the retracted position inside the outer cylinder to cause the support portion to move to the extended position in which the support portion extends distally beyond the distal-end opening of the outer cylinder.

2. The transfer instrument according to claim 1, wherein the support portion has sides spaced apart from one another in the width direction, the support portion being configured to hold the medical sheet so as to bring portions of the medical sheet protruding from both sides of the support portion into contact with each other while the support portion is in the retracted position in the outer cylinder and deformed into the curved shape.

3. The transfer instrument according to claim 2, wherein the support portion is configured to hold the medical sheet so as to bring the portions of the medical sheet protruding from both the sides of the support portion into contact with a portion of the medical sheet supported by the support surface while the support portion is in the retracted position in the outer cylinder and deformed into the curved shape.

4. The transfer instrument according to claim 1, wherein the support portion has a corrugated shape in cross section along the width direction of the support portion when the support portion is in the extended position extending distally beyond the distal-end opening of the outer cylinder, and
when the support portion is in the retracted position in the outer cylinder and deformed into the curved shape, at least a part of the corrugated shape of the support portion is curved to bulge toward an inside of the outer cylinder.

5. The transfer instrument according to claim 1, wherein the support portion has sides spaced apart from one another in the width direction, each of the sides of the support portion including a protrusion that projects upwardly away from the support surface when the support portion is horizontal and the support surface is facing upwardly.

6. A transfer instrument for transferring a medical sheet to a treatment site of a living body, the transfer instrument comprising:
an outer cylinder having a through hole that extends from a distal-end opening at a distal end of the outer cylinder to a proximal-end opening at a proximal end of the outer cylinder;
a carrier member that includes a shaft and a support portion, the shaft having a distal end portion and the support portion having a proximal-end support portion and a distal-end support portion, the proximal-end support portion terminating at a connection joint that is fixed to the distal end portion of the shaft so that movement of the shaft results in movement of the support portion, the shaft having a central axis;
the shaft being positionable in the through hole in the outer cylinder and being axially movable in an axial direction relative to the outer cylinder to move the support portion from an extended position in which the support portion is distal of the distal-end opening of the outer cylinder with the medical sheet supported on the support portion to a retracted position in which the support portion and the medical sheet are retracted into the through hole in the outer cylinder by axially moving the shaft in a proximal direction relative to the outer cylinder, and being axially movable from the retracted position to the extended position by moving the shaft in a distal direction relative to the outer cylinder; and
the support portion having a width orthogonal to the axial direction, the width of the support being greater than the distal-end opening at the distal end of the outer cylinder so that as the support portion is moved from the extended position to the retracted position the support portion contacts the distal end of the outer cylinder and is deformed into a curved shape together with the medical sheet.

7. The transfer instrument according to claim 6, wherein the proximal-end support portion has a tapered shape that tapers towards the connection joint, the tapered shape contacting the distal end of the outer cylinder as the support portion is moved from the extended position to the retracted position to assist the deformation of the support portion and the medical sheet into the curved shape.

8. The transfer instrument according to claim 7, wherein the proximal-end support portion has the tapered shape on opposite sides of the axial direction.

9. The transfer instrument according to claim 6, wherein the support portion has sides spaced apart from one another in the width direction, each of the sides of the support portion including a protrusion that projects upwardly away from the support surface when the support portion is horizontal and the support surface is facing upwardly.

10. The transfer instrument according to claim 9, wherein the protrusions are distal of the proximal-end support portion that has the tapered shape.

11. The transfer instrument according to claim 6, wherein the support portion has sides spaced apart from one another in the width direction, the support portion being configured to hold the medical sheet so as to bring portions of the medical sheet protruding from both sides of the support portion into contact with each other while the support portion is in the retracted position in the outer cylinder and deformed into the curved shape.

12. The transfer instrument according to claim 11, wherein the support portion is configured to hold the medical sheet so as to bring the portions of the medical sheet protruding from both the sides of the support portion into contact with a portion of the medical sheet supported by the support surface while the support portion is in the retracted position in the outer cylinder and deformed into the curved shape.

13. The transfer instrument according to claim 6, wherein the support portion has a corrugated shape in cross section along the width direction of the support portion when the support portion is in the extended position extending distally beyond the distal-end opening of the outer cylinder, and when the support portion is in the retracted position in the outer cylinder and deformed into the curved shape, at least a part of the corrugated shape of the support portion is curved to bulge toward an inside of the outer cylinder.

14. A method comprising:
moving a support portion toward a dish on which is located a medical sheet, the support portion being fixed to a distal end portion of a shaft and the shaft being positioned in an outer cylinder so that a proximal end of the shaft extends proximally beyond a proximal end of the outer cylinder and the support portion is in an extended position in which the support portion extends distally beyond a distal-open end at a distal end of the outer cylinder, the support portion including a support surface and an oppositely facing back surface;
placing the medical sheet on the support surface while the support portion is in the extended position, the placing of the medical sheet on the support surface including placing the medical sheet on the support surface such that a portion of the medical sheet extends outwardly beyond an outer periphery of the support portion; retracting the support portion together with the medical sheet into the outer cylinder by moving the support portion relative to the outer cylinder from the extended position to a retracted position in which the support portion and the medical sheet are located inside the outer cylinder;
during the retracting of the support portion and the medical sheet into the outer cylinder, the support portion being deformed into a curved shape and a part of the portion of the medical sheet extending outwardly beyond the outer periphery of the support portion contacting another part of the medical sheet.

15. The method according to claim 14, further comprising moving the outer cylinder toward a treatment site of a living body to which the medical sheet is to be applied, the moving of the outer cylinder toward the treatment site of the living body occurring while the support portion and the medical sheet are in the retracted position in the outer cylinder.

16. The method according to claim 14, further comprising advancing the support portion together with the medical sheet from the retracted position to the extended position so that the support portion and the medical sheet change from the curve shape to a flat shape allowing the medical sheet to be removed from the support portion.

17. The method according to claim 14, wherein the retracting of the support portion together with the medical sheet into the outer cylinder includes retracting the support portion together with the medical sheet into the outer cylinder so that a distal end of the support portion is proximal of the distal-open end of the outer cylinder.

18. The method according to claim 14, wherein the support portion has sides spaced apart from one another in a width direction of the support portion that is orthogonal to an axial direction of the outer cylinder, the portion of the medical sheet that extends outwardly beyond the outer periphery of the support portion including side portions of the medical sheet that extend outwardly beyond the sides of the support portion, the part of the portion of the medical sheet that contacts the another part of the medical sheet including the side portions of the medical sheet.

19. The method according to claim 14, wherein the support portion has sides spaced apart from one another in a width direction of the support portion that is orthogonal to an axial direction of the outer cylinder, the portion of the medical sheet that extends outwardly beyond the outer periphery of the support portion including side portions of the medical sheet that extend outwardly beyond the sides of the support portion, the medical sheet including a supported portion that is supported by the support surface of the support portion when the support portion is in the extended position and that is located between the side portions of the medical sheet, the side portions of the medical sheet contacting the supported portion of the medical sheet when the support portion is in the retracted position in the outer cylinder.

20. The method according to claim 14, wherein the support portion has a corrugated shape in cross section along a width direction of the support portion that is orthogonal to an axial direction of the outer cylinder when the support portion is in the extended position, extending distally beyond the distal-end opening of the outer cylinder, at least a part of the corrugated shape of the support portion being curved to bulge toward an inside of the outer cylinder when the support portion is in the retracted position in the outer cylinder.
